(19) United States
(12) Patent Application Publication
(10) Pub. No.: US 2026/0092931 A1
(43) Pub. Date: Apr. 2, 2026

(54) Bcl2 Family in Dysfunctional Neurons Is Critical to the Evolution, Diagnosis and Treatment of Neurodegenerative Diseases Including but Not Limited to Corticobasal Degeneration, Chronic Traumatic Encephalopathy, Amyotrophic Lateral Sclerosis (Als), Alzheimer's Disease, Parkinson's Disease, Down's Syndrome Dementia, and Lewy Body Dementia

(71) Applicant: Gerard Nuovo, Westerville, OH (US)

(72) Inventor: Gerard Nuovo, Westerville, OH (US)

(21) Appl. No.: 18/233,947

(22) Filed: Aug. 15, 2023

(51) Int. Cl. G01N 33/68; A61K 45/06; C07K 16/32; G01N 33/50

(52) U.S. Cl. CPC G01N 33/6896; A61K 45/06; C07K 16/32; G01N 33/5058; G01N 2333/4704; G01N 2800/2814

(57) ABSTRACT

Diagnostic and therapeutic methods for neurodegenerative diseases. Are provided involving assaying abnormal proteins (hyperphosphorylated tau, α-synuclein, TDP-43) associated with neuronal turnover inhibition or promotion in patient samples. Abnormal protein expression and apoptotic activity are detected, aiding disease progression assessment. Therapeutically, a method is provided for treating neurodegenerative diseases, administering compounds promoting neuronal turnover or modulating proteins involved in the process. The invention extends to identifying suitable drugs, employing neuronal turnover induction, miRNA modulation, and protein activity inhibition or enhancement. The claims also encompass various species, tissues, and cultured cells. Furthermore, the invention is applicable to diverse neurodegenerative diseases with abnormal protein accumulation, presenting novel diagnostic and treatment approaches.

### RELATED APPLICATIONS

The present invention extends the original and unexpected findings of Provisional application No. 61/981,2376 and 62/341,259, incorporated by reference as if fully rewritten herein. There are no previously filed, nor currently any co-pending applications, anywhere in the world.

### STATEMENT REGARDING FEDERALLY SPONSORED RESEARCH

Not applicable.

### BACKGROUND OF THE INVENTION

### 1. Field of the Invention

The present invention relates generally to the treatment of neurodegenerative diseases and, more particularly, to the novel and original discovery that the underlying mechanism in neurodegenerative case is that the neurons with these abnormal proteins are incapable of neuronal turnover which is the process whereby old neurons die and are replaced by new, healthy neurons.

### 2. Description of the Related Art

Each of the neurodegenerative diseases is marked by the accumulation of an abnormal protein in neurons that defines the disease. These abnormal proteins make the neurons dysfunctional. In Alzheimer's disease, chronic traumatic encephalopathy, and corticobasal degeneration the abnormal protein is hyperphosphorylated tau protein, in Lewy body dementia and Parkinson's disease it is α-synuclein, and in ALS as well as frontotemporal dementia it is TDP-43. It is now found that the underlying mechanism in each case is that the neurons with these abnormal proteins are incapable of neuronal turnover which is the process whereby old neurons die and are replaced by new, healthy neurons. The reason that they are incapable of neuronal turnover is because they have accumulated one or more proteins which block neuronal turnover. These “anti-neuronal turnover” proteins include the so-called bcl-2 family (bcl2, bclW, bclXL, MCL1) as well as cFLIP. This novel discovery has major implications for the diagnosis, monitoring, and treatment of neurodegenerative diseases. If one or more of these proteins are blocked, the diseased neurons can die and be replaced by new, healthy neurons.

The three most common neurodegenerative diseases in the USA are Alzheimer's disease, Parkinson's disease, and amyotrophic lateral sclerosis (ALS). Other relatively common neurogenerative diseases include corticobasal degeneration (CBD), frontotemporal dementia (FTD), chronic traumatic encephalopathy (CTE), and Lewy body dementia (LBD). Each disease is marked by the accumulation of an abnormal protein in neurons that shares several features in common: 1) the abnormal protein came from a normal neuronal protein; 2) the normal protein was altered, including being hyperphosphorylated and, 3) the abnormal protein can be detected in situ and serves as the diagnostic feature of the disease, and 4) the abnormal protein induces an inflammatory response which increases the dysfunction in the neurons. There are three such proteins. 1) Hyperphosphorylated tau protein is the most common and is the diagnostic feature of Alzheimer's disease, CTE, Down's syndrome dementia, and CBD. These diseases also share another abnormal protein that is derived from a normal brain protein called amyloid precursor protein. The abnormal protein is called amyloid-β. However, unlike hyperphosphorylated tau protein which in our extensive experience is not found in brain tissues from elderly people who are not demented, it is also found in the brain of people who do not have dementia. 2) TDP-43 which is the abnormal protein in the large motor neurons of ALS and is also found in FTD. 3) a synuclein which is the abnormal protein in LBD and PD. In each case the hyperphosphorylated tau protein is dysfunctional. For example, the neuronal isoforms of tau protein have about 80 serine and threonine phosphorylation sites. It has been well documented that phosphorylation of less than 50% of these sites invariably is present in normal brain and that phosphorylation of >75% of these sites is associated with the loss of function of tau. This hyperphosphorylated tau protein shows increased intracellular precipitation and is easily detected by immunohistochemistry using antibodies that cannot detect the normal tau protein.

Thus, finding the abnormal tau, or TDP-43 as well as α-synuclein is a simple way to easily find neurons that cannot be functioning normally. Each of these dysfunctional neurons connects via synapses to hundreds/thousands of other neurons either directly or indirectly. It is a simple but important point to stress that when these normal neurons do not get signals from the dysfunctional neurons, then also will not function normally that could lead to their own cell death.

Each of the neurodegenerative diseases is marked by the loss of large numbers of neurons (called brain atrophy) in the brain at their end stage. Not surprisingly, many researchers have speculated that the neurons with the abnormal proteins diagnostic of the given disease have an increased rate of cell death (eg apoptosis) that in turn leads to the brain atrophy typical of the disease. A widely held view is that an increased rate of apoptosis may be triggered by the more rapid accumulations in neurons of the abnormal proteins noted above due to the toxicity associated with the protein per se and the inflammation that it induces. To this point, various investigators have noted that classic apoptotic proteins such as caspase-3, caspase-8, and apoptosis initiating factor can be found either directly in the neurons with hyperphosphorylated tau protein, α-synuclein, TDP-43, or in the microglial cells that surround such cells. It has been theorized that these proteins can accumulate at an increased rate in neurons and that increased accumulation would be expected to inevitably lead to the apoptotic death of these hyper-accumulating cells. More directly, various studies have presented evidence of elevated intrinsic and extrinsic apoptotic pathways in Alzheimer's disease either in the neurons per se or in the associated support cells including microglial.

It is now clear that the central nervous tissue, like all organs, undergoes a normal turnover of cells including neurons. For some tissues this turnover rate is well defined; skin typically completely renews itself every 39 days whereas red blood cells renew themselves every 120 days. The rate of neuronal turnover in the brain is not known but may be similar to other organs such as the bone that replaces about 2% of its primary cell (osteoblast) per year. Critically, Spalding et al (Dynamics of hippocampal neurogenesis in adult humans. Cell. 2013 Jun. 6; 153(6):1219-1227. doi: 10.1016/j.cell.2013.05.002. PMID: 23746839; PMCID: PMC4394608) found that “a large subpopulation of hippocampal neurons, constituting one third of the neurons, is subject to exchange. In adult humans, 700 new neurons are added per day, corresponding to an annual turnover of 1.75% of the neurons within the renewing fraction.” Several proteins are known to be phenotypic markers of neuronal progenitor cells including MCM2 and nestin. It has been established using phenotypic markers of neuronal progenitor cells that there are conditions, such as epilepsy, where the brain responds with increased mature neuronal production. This invites the question as to whether some neurodegenerative diseases may reflect DECREASED neuronal cell turnover.

Most studies on the molecular biology of AD, CBD, CTE, PD, ALS, LBD, and other dementias extract brain tissues and do the analyses. Although useful, such data cannot pinpoint changes in specific cell types in general and in the neurons that contain the abnormal proteins diagnostic of the dementia in particular. In situ-based methods solve this problem since the tissue is completely intact during analyses and it is straightforward to do co-expression analyses and determine if a given cell contains multiple proteins and/or RNAs.

Treatments against Alzheimer's disease have focused on removing either the hyperphosphorylated tau protein or amyloid-β protein. Although antibodies directed against these two proteins can be documented to reduce the burden in the brain of said protein in people with Alzheimer's disease, the clinical benefits have been disappointing, with a much less clinical benefit relative to the change in the abnormal protein burden. This suggests that removing the amyloid-β protein may not be addressing the disease process that is at the epicenter of Alzheimer's disease in particular and the other neurodegenerative diseases in general.

### SUMMARY OF THE INVENTION

Neurogenerative diseases are due to the dysregulation of neuronal cell turnover which, in turn, are based on a relatively high ratio of anti-apoptotic proteins (bcl2-family, cFLIP) to the apoptotic proteins (bcl2 family including BIM, BAD, BAK, BAX, NOXA, PUMA). It is thus an object of the present invention for the blocking of an anti-apoptotic protein(s) allows the dysfunctional neurons to be replaced (turnover) by new, healthy neurons.

Briefly described according to the preferred embodiment, the present invention extends the original and unexpected findings of my provisional patent EFS ID: 18803361), where the importance of the anti-apoptotic proteins MCL1 (a member of the bcl2 family) and cFLIP in the essential pathology of Alzheimer's disease was stressed. This work has been extended to include other members of the bcl2 family that include bclXL, bcl2, bclW, BAD, BIM, BAK, BAX, PUMA, plus NOXA and their modulators p16 and p53. Critically, this extension now applies to all neurodegenerative diseases in that I have documented that the same strong correlation of the bcl2 family and cFLIP found with hyperphosphorylated tau protein in Alzheimer's disease also applies to the Lewy body (α-synuclein) in PD and LBD as well as to hyperphosphorylated tau protein in CBD as well as to TDP-43 and ALS. I have also generated experimental data using a mouse model of Alzheimer's disease (Tau4RΔK (CamKII-tTA;TetO-TauRDΔK) which contains the human hyperphosphorylated tau protein. These mice develop symptoms of Alzheimer's disease as early as 4 months of age and usually die after 1 year of the disease. However, by simply treating these mice with an anti-bcl2 drug (venetoclax) they have shown no signs of the disease whereas their untreated litter mates already are showing the classic disease state at about 4 months of age.

The present disclosure opens wide new avenues for understanding and treating neurogenerative diseases in general by indicating that the loss of the normal neuronal cell turnover due to the predominance of anti-apoptotic factors is a causative agent of the disease, or correlated with the disease. Also presented are data that strongly suggests a similar process may apply to other neurodegenerative diseases, including ALS, LBD, CBD, Parkinson's disease and by extension CTE as well as FTD. Thus, the abnormal longevity of affected neurons is a primary factor responsible for the accumulation of the increased pathologic markers that characterize the dysfunctional neurons and, critically, this process can be reversed by tipping the anti-neuronal cell turnover/neuronal cell turnover balance in favor of the latter.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1A through 1C depict the expression of proteins that can block neuronal turnover in the brain. Note the increased expression of bcl2, bclX, bclXL, MCL1 and cFLIP in the mouse with human abnormal tau protein and how these proteins closely parallel the presence of the human hyperphosphorylated tau protein.

FIG. 2A through 2D depict the expression of proteins that can allow neuronal turnover in the brain. Note the increased expression of p53, BAD, BIM, NOXA, PUMA, BAK, BAX, and p16 in the mouse with human abnormal tau protein and how these proteins closely parallel the presence of the human hyperphosphorylated tau protein.

FIG. 3 shows the bcl2 family and cFLIP localize to neurons with human hyperphosphorylated tau protein in the Tau4RΔK mouse model of Alzheimer's disease (BAK and tau (A-C), bcl2 and neuronal marker (D-F), cFLIP and neuronal marker (G), bclW and neuronal marker (H), and BAX and tau (I). Co-expression is evident in all cases by fluorescent yellow.

FIG. 4 shows Hyperphosphorylated tau protein in humans with Alzheimer's disease localizes to neurons; Tau (A), astrocyte GFAP (B), neuronal marker (C), microglia (TMEM) (D), strong co-localization of tau in neurons (E, yellow=tau in neurons), lack of tau in astrocytes (F, no yellow co-localization).

FIG. 5 shows increased expression of proteins that block neuronal cell turnover (bcl2, bclW, bclXL, MCL1 and cFLIP) as well as facilitate neuronal turnover (BAD, BIM, and BAX) in CBD, ALS, LBD, and Parkinson's disease in humans.

FIG. 6 shows co-localization of proteins that either block or enhance cell turnover in neurons in patients with ALS. A-C=bclXL (A), TDP-43 (B), and co-localization seen as yellow (C). D-F=BIM (D), TDP-43 (E), and co-localization seen as yellow (F). G-I=TDP-43 (G), neuronal marker (H), and co-localization seen as yellow (I).

FIG. 7 shows co-localization of proteins that either block or enhance cell turnover in neurons in patients with CBD. A-C=tau (A), MCL1 (B), and co-localization seen as yellow (C). D-F=MCL1 (D), neuronal marker (E), and co-localization seen as yellow (F). G-I=tau (G), bclW (H), and co-localization seen as yellow (I).

FIG. 8 depicts co-localization of proteins that either block or enhance cell turnover in neurons in patients with LBD. A-C=α-synuclein (A), neuronal marker (B), and co-localization seen as yellow (C). D-F=cFLIP (D), MCL1 (E), and co-localization seen as yellow (F). G-I=bcl2 (G), BIM (H), and co-localization seen as yellow (I).

FIG. 9 depicts co-localization of proteins that either block or enhance cell turnover in neurons in patients with PD. A-C=α-synuclein (A), BIM (B), and co-localization seen as yellow (C). D-F=α-synuclein (D), MCL1 (E), and co-localization seen as yellow (F). Panel G is a Western blot of MCL1 from two normal brains (NL) and five cases of PD. Panel H is a Western blot of cFLIP from two normal brains (NL) and three cases of PD. Panel I is a Western blot of BIM from eight cases of PD.

FIG. 10 depicts co-localization of MCL1 mRNA (A), MCL1 protein (B), BIM protein (C) and a neuronal marker (D) in a patient with PD. Note in panel E the fluorescent yellow indicative of neurons expressing MCL1 RNA, protein and BIM.

The patent or application file contains at least one drawing executed in color. Copies of this patent or patent application publication with color drawing(s) will be provided by the Office upon request and payment of the necessary fee.

### DETAILED DESCRIPTION OF THE PREFERRED EMBODIMENTS

The present invention is based upon a comprehensive and novel examination of the following question: is there any molecular process that links the various neurodegenerative diseases (including AD, CBD, CTE, Down's syndrome dementia, PD, LBD, FTD, and ALS) that can be exploited in the diagnosis, monitoring, and treatment of these disparate diseases?

It is important in the understanding of the present invention to consider the my group's prior extensive publications on the molecular biology of dementia, with a focus on Alzheimer's disease and Down's syndrome dementia. Here is a list of the relevant citations with a brief summary of each paper:
1. Mezache L, Mikhail M, Garofalo M, Nuovo G J. Reduced miR-512 and the Elevated Expression of Its Targets cFLIP and MCL1 Localize to Neurons with Hyperphosphorylated Tau Protein in Alzheimer Disease. Appl Immunohistochem Mol Morphol. 2015 October; 23 (9): 615-23. doi: 10.1097/PAI.0000000000000147. PMID: 26258756. (the first demonstration that two key proteins that block cell turnover—MCL1 and cFLIP—are found in neurons with hyperphosphorylated tau protein in Alzheimer's disease).
2. Nuovo G, Paniccia B, Mezache L, Quiñónez M, Williams J, Vandiver P, Fadda P, Amann V. Diagnostic pathology of Alzheimer's disease from routine microscopy to immunohistochemistry and experimental correlations. Ann Diagn Pathol. 2017 June; 28:24-29. doi: 10.1016/j.anndiagpath.2017.02.006. Epub 2017 Feb. 13. PMID: 28648936. (we demonstrated that the neurons with hyperphosphorylated tau protein were viable and were able in neuronal cell culture to induce MCL1 and cFLIP and show these neurons accumulated hyperphosphorylated tau protein).
3. Nuovo G, Amann V, Williams J, Vandiver P, Quinonez M, Fadda P, Paniccia B, Mezache L, Mikhail A. Increased expression of importin-β, exportin-5 and nuclear transportable proteins in Alzheimer's disease aids anatomic pathologists in its diagnosis. Ann Diagn Pathol. 2018 February; 32:10-16. doi: 10.1016/j.anndiagpath.2017.08.003. Epub 2017 Sep. 7. PMID: 29414391. (we further demonstrated that the neurons with hyperphosphorylated tau protein were viable and had increased nuclear transport compared to normal neurons).
4. Tili E, Mezache L, Michaille J J, Amann V, Williams J, Vandiver P, Quinonez M, Fadda P, Mikhail A, Nuovo G. microRNA 155 up regulation in the CNS is strongly correlated to Down's syndrome dementia. Ann Diagn Pathol. 2018 June; 34:103-109. doi: 10.1016/j.anndiagpath.2018.03.006. Epub 2018 Mar. 26. PMID: 29661714. (we demonstrated that Down's syndrome dementia had some unique features compared to Alzheimer's disease; although they both showed hyperphosphorylated tau protein the former had decreased neuronal proteins related to the high miR 155 burden. Importantly, we also showed that neuron cells in culture could be induced to express more MCL1 and that this was associated with increased bcl2 and BAK).
5. Nuovo G J, Suster D, Sawant D, Mishra A, Michaille J J, Tili E. The amplification of CNS damage in Alzheimer's disease due to SARS-COV2 infection. Ann Diagn Pathol. 2022 December; 61:152057. doi: 10.1016/j.anndiagpath.2022.152057. Epub 2022 Oct. 28. PMID: 36334414; PMCID: PMC9616485. (we showed that the SARS-COV2 virus causes a microencephalitis in the areas with hyperphosphorylated tau protein which gave a molecular basis for the worsening of Alzheimer's disease in people with COVID-19.
   The above work has been much more elucidated as detailed in this grant application. This extra work can be divided into two groups: 1) mouse models of Alzheimer's disease and 2) human neurodegenerative diseases (AD, LBD, CBD, ALS, and PD).

### Mouse Models of AD

My group obtained genetically modified mice that were able to express in the brain the HUMAN hyperphosphorylated tau protein or the HUMAN amyloid-β protein (ie Aβ-42); in the latter case we obtained two genetically different types of mice. These mice each develop large amounts of either hyperphosphorylated tau protein or Aβ 42 in distinct regions of their brain that closely mimic the pattern in human AD brains and eventually develop the dementia and in cases die of the disease. This offers an opportunity not available with human Alzheimer's disease tissues; one can molecularly separate out the effects of hyperphosphorylated tau protein and the Aβ 42 protein. The paper (now resubmitted as a revised manuscript) is also provided in the Information Disclosure Statement as reference Cite No. AA. The ABSTRACT of the paper follows:

### ABSTRACT

Both neurofibrillary tangles and senile plaques are associated with inflammation in Alzheimer's disease (AD). Their relative degree of induced neuroinflammation, however, is not well established. Mouse models of AD that expressed either human Aβ42 (n=7) or human hyperphosphorylated tau protein alone (n=3), wild type (n=10) and human AD samples (n=29 with 18 controls) were studied. The benefit of using mouse models that possess only human tau or amyloid-β is that it allows for the individual evaluation of how each protein affects neuroinflammation, something not possible in human tissue. Three indicators of neuroinflammation were examined: TLRs/RIG1 expression, the density of astrocytes and microglial cells, and well-established mediators of neuroinflammation (IL6, TNFα, IL1β, and CXCL10). There was a statistically significant increase in neuroinflammation with all three variables in the mouse models with human tau only as compared to human Aβ42 only or wild type mice (each at p<0.0001). Only the Aβ42 5×FAD mice (n=4) showed statistically higher neuroinflammation versus wild type (p=0.0030). The human AD tissues were segregated into Aβ42 only or hyperphosphorylated tau protein with Aβ42. The latter areas showed increased neuroinflammation with each of the three variables compared to the areas with only Aβ42. Of the TLRs and RIG-1, TLR8 was significantly elevated in both the mouse model and human AD and only in areas with the abnormal tau protein. It is concluded that although Aβ42 and hyperphosphorylated tau protein can each induce inflammation, the latter protein is associated with a much stronger neuroinflammatory response vis-α-vis a significantly greater activated microglial response.
   On information and belief, this is the first time that it has been documented that hyperphosphorylated tau protein induces a stronger neuroinflammation than Aβ42 protein.

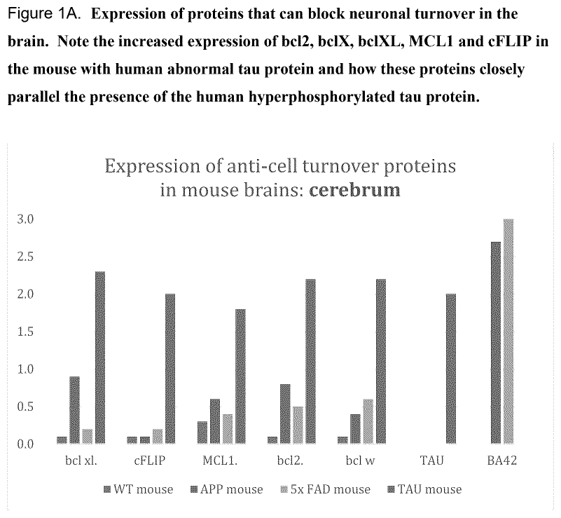

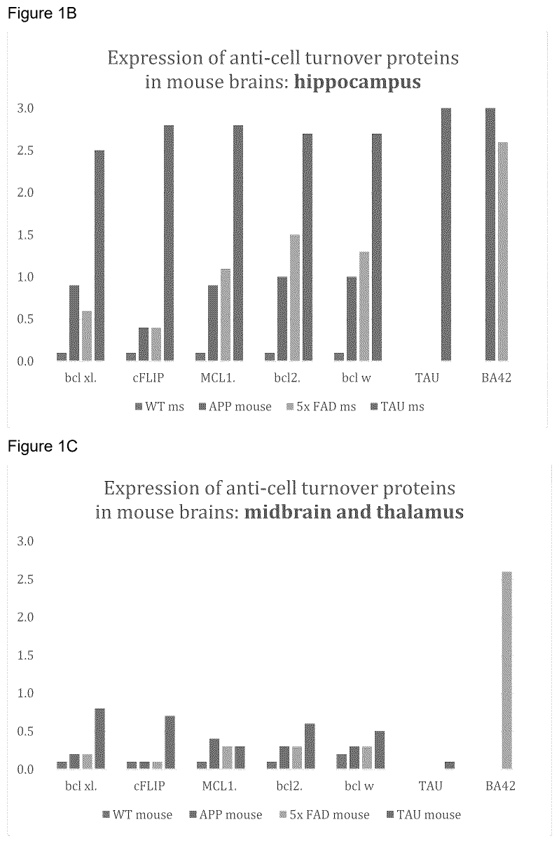

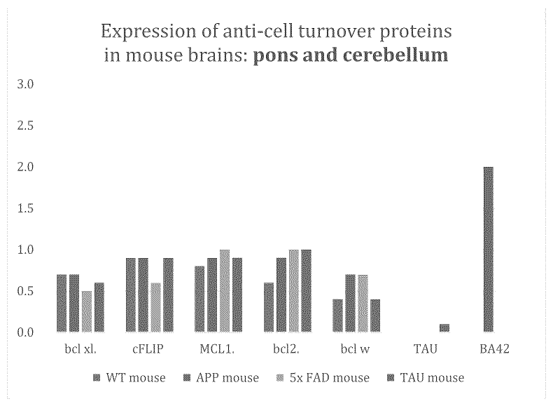

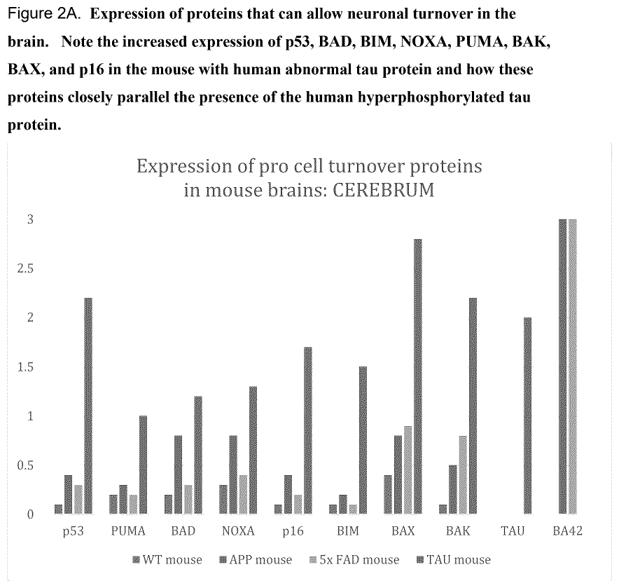

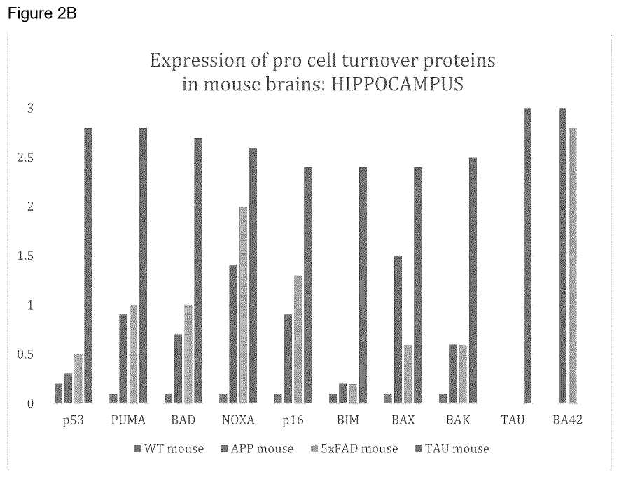

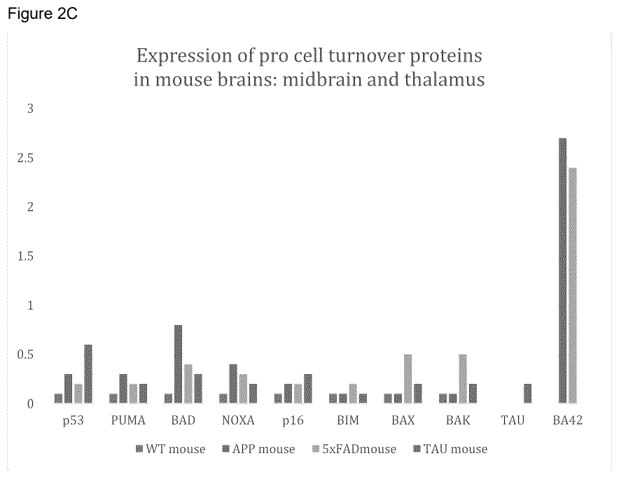

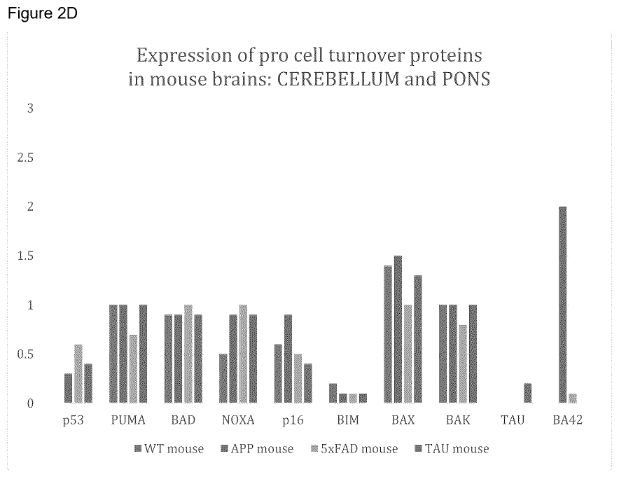

Our group also answered the question: is the bcl2 family of proteins as well as cFLIP correlated with either hyperphosphorylated tau protein and/or Aβ42 protein in these mouse models of AD? The answer was YES for hyperphosphorylated tau protein and NO for Aβ42 protein. These data are presented in graphic form (FIG. 1 shows the proteins that inhibit neuronal turnover and FIG. 2 the proteins that can induce neuronal turnover. Note that each of the bcl2 family proteins that can block cell/neuronal turnover (bcl2, bclW, bclXL, MCL1) as well as cFLIP are markedly increased in the areas where the abnormal human hyperphosphorylated tau protein is present. Similarly, the bcl2 family members that can assist a neuron in turnover (NOXA, PUMA, BIM, BAD) as well as related proteins p53 and p16 are also increased in these hyperphosphorylated tau protein rich areas. The ratio of these two groups of proteins is what I am referring to as the anti-neuronal turnover/pro-neuronal turnover balance. The key to disrupting this balance and allowing the neuron to enter neuronal cell turnover is to remove one or more of the former (bcl2, bclW, bclXL, MCL1, or cFLIP).

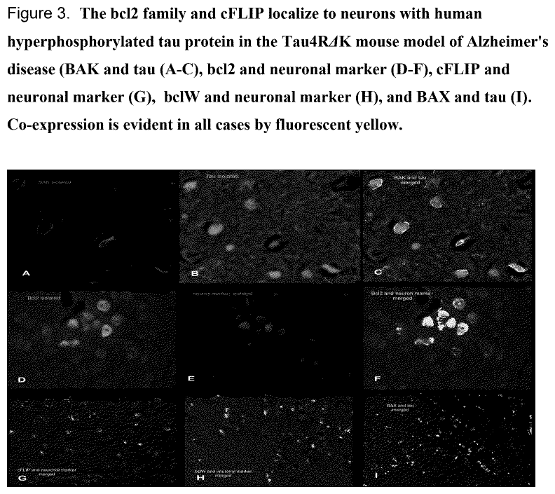

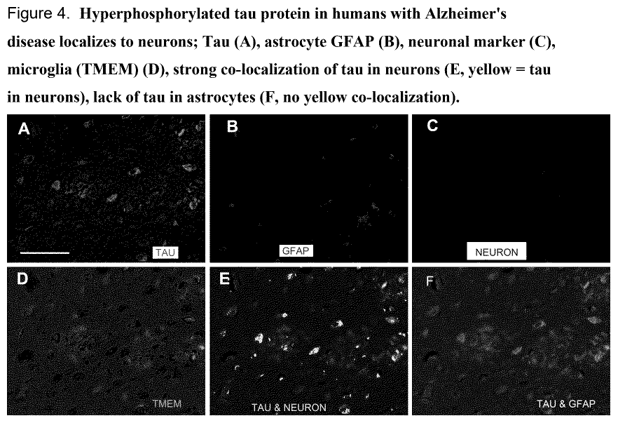

Co-expression analyses allowed our group to answer the next simple but critical question: are these apoptotic and anti-apoptotic proteins located in neurons or the supporting glial cells? The answer was that over 90% of the cells that contained these proteins were neurons. Critically, they were not just neurons but more specifically neurons that also contained hyperphosphorylated tau protein. This is illustrated in FIG. 3 using co-expression for hyperphosphorylated tau protein and a neuronal marker (pyruvate dehydrogenase or NeuN—illustrated is the former) for a variety of bcl2 family proteins and cFLIP. FIG. 4 used co-expression for 4 targets (tau, a neuronal marker, and markers of microglial cells (TMEM 119) as well as astrocytes (GFAP)) to demonstrate that the hyperphosphorylated tau protein is localizing primarily in neurons in human disease.

In sum, the data from the mouse AD models clearly demonstrated that the bcl2 family members studied including bcl2, bclW, bclXL, MCL1, BAD, BIM, NOXA, PUMA, BAK, and BAX, as well as cFLIP, p53, and p16 are all strongly correlated with the murine neurons that are expressing the human hyperphosphorylated tau protein but not Aβ-42 AND that this was, in turn, was strongly correlated to the degree of neuroinflammation which clearly would be synergistic with the abnormal tau protein per se to cause the neurologic symptoms, brain atrophy, and resultant death that inevitably occurs in these mice.

### Human AD, LBD, CBD, ALS, and PD and the Bcl2-Family Proteins in the Abnormal Neurons

The next set of experiments focused on four other neurodegenerative diseases in human tissues: CBD, ALS, LBD, and PD. Each of these diseases is diagnosed using a combination of clinical and laboratory results and an essential part of the diagnosis is post-mortem brain or spinal cord tissue in which the diagnostic immunohistochemistry based abnormal protein can be identified. With CBD this is hyperphosphorylated tau protein, with ALS it is TDP-43, and with LBD and PD it is α-synuclein. Our group studied 4 cases of CBD, 20 of ALS, 4 of LBD, and 16 cases of PD. Included were 10 aged matched control brains from people who had no history or evidence of any neurodegenerative disease. The data were analyzed blinded to the clinical and pathologic diagnosis. In none of the controls were either hyperphosphorylated tau protein, TDP-43, or α-synuclein evident. In each of the 4 cases of CBD, 4 cases of LBD, and 20 cases of ALS the diagnostic abnormal hyperphosphorylated protein was detected. In 12/16 of the PD cases, α-synuclein was evident and, thus, only these were studied further.

Each tissue was examined for bcl2-family proteins that can block neuronal turnover (bcl2, bclW, bclXL, MCL1) as well as cFLIP. Each tissue was also tested for the bcl-2 family of proteins that can induce neuronal cell turnover including BIM, BAD, and BAX.

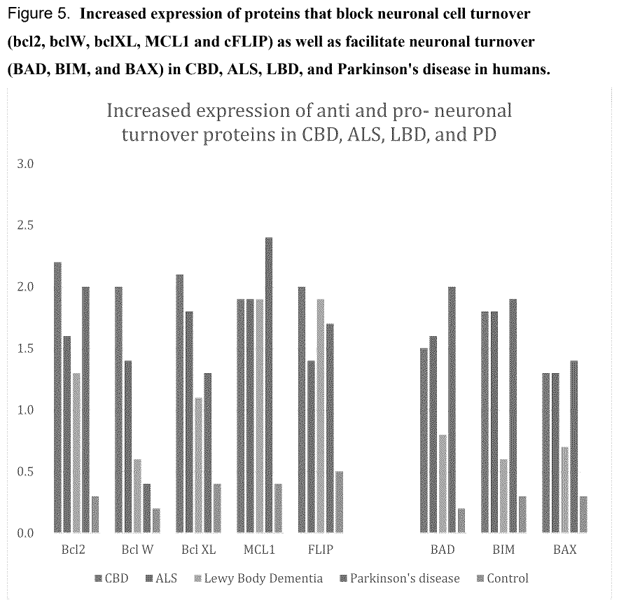

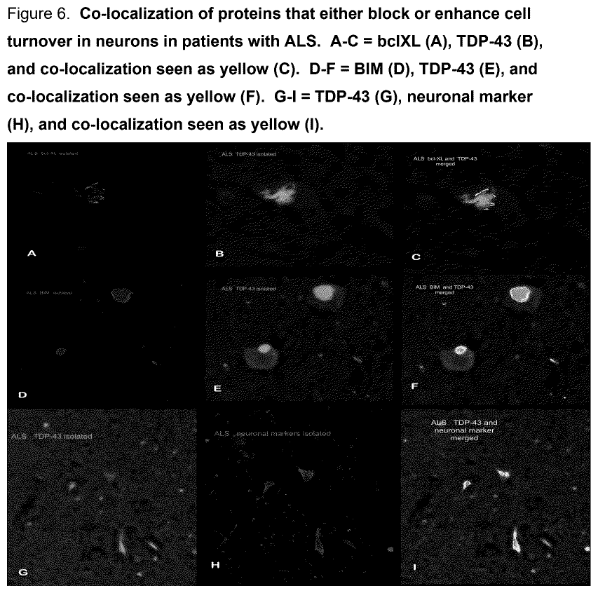

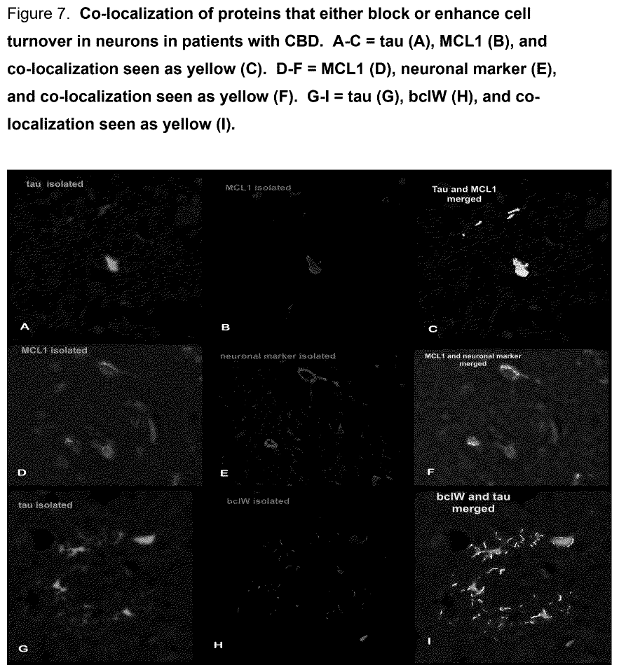

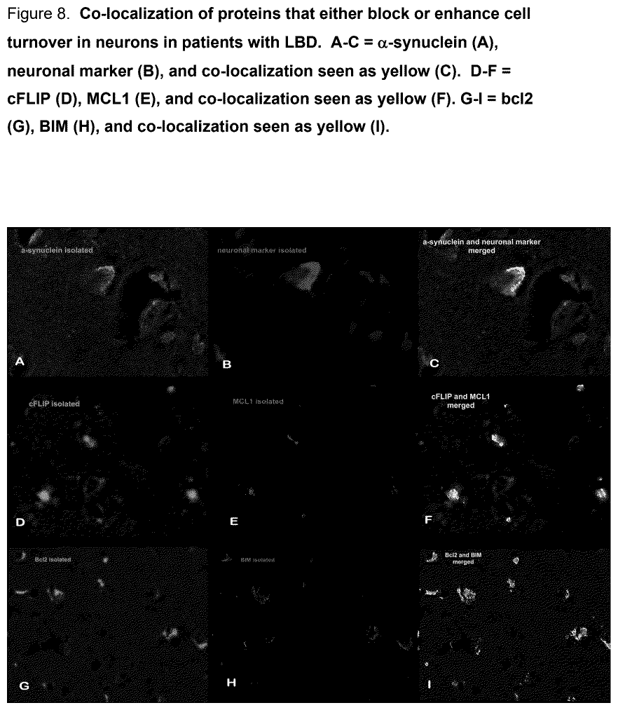

The key finding for each disease category was unexpected, novel, and the foundation of this patent application: in each case, there was marked increase of each of the proteins that can block neuronal turnover as well as each of these proteins that can induce neuronal turnover. As evident in FIG. 5, these proteins (bcl2, bclW, bclXL, MCL1, cFLIP, BIM, BAD, and BAX) are typically highly expressed in the brain tissues of people who died of CBD, ALS, LBD, and PD in sections which contain easily detectable abnormal proteins diagnostic of the specific disease. Critically, as also evident from the co-expression data in FIGS. 6 (ALS), 7 (CBD), 8 (LBD), and 9 (PD), the vast majority of cells which contained these proteins were neurons that also expressed hyperphosphorylated tau protein (CBD as well as, of course, AD), TDP-43 (ALS), or α-synuclein (LBD and PD). These data strongly suggest the unexpected and novel finding that these neurodegenerative diseases may have as their fundamental pathogenesis a failure of normal neuronal turnover, where aged, “immortalized” neurons are blocked from turnover, amass the characteristic abnormal protein, induce neuroinflammation, and directly lead to the malfunction and finally death of the innumerable neurons that are connected to these dysfunctional neurons. Clearly, these data have strong implications for the diagnosis of neurodegenerative diseases since early detection of these apoptotic and anti-apoptotic proteins could predict the disease state, even before the abnormal hyperphosphorylated proteins are abundant. But perhaps more importantly they have especially important implications in the treatment of these many disparate neurodegenerative diseases, as they strongly suggest that inhibiting either bcl2, bclW, bclXL, MCL1, and/or cFLIP may allow the dysfunctional neurons to re-enter the neuronal turnover pool and be replaced by new, healthy neurons.

The unexpected findings in this patent application are several-fold. First, it is controversial if neurons or other cell types (specifically microglial cells and astrocytes) contain the diagnostic proteins (hyperphosphorylated tau protein, TDP-43, or α-synuclein). This is one of the reasons that the co-expression images contain multiple examples documenting that it is the neuron which by far is the predominant cell that contains the abnormal proteins diagnostic of the specific neurodegenerative disease. Indeed, these neurons also contain the bcl2 family proteins (pro and anti-apoptotic). Another unexpected finding is the role of bcl2, bclW, bclXL, MCL1, cFLIP in neurodegenerative diseases in general. The relatively scarce data often suggests that these proteins may protect neurons from death and, thus, may be protective against neurodegenerative diseases. However, the data in this patent application strongly suggests the opposite is true and that it is the blocking of these proteins that may be the most direct and effective way to both halt as well as revert the disease process.

There have been several papers recently that have introduced the term of senescence with neurodegenerative diseases. One such paper (Bussian T J, et al. Clearance of senescent glial cells prevents tau-dependent pathology and cognitive decline. Nature. 2018 October; 562 (7728): 578-582. doi: 10.1038/s41586-018-0543-y. Epub 2018 Sep. 19. PMID: 30232451; PMCID: PMC6206507) used a drug which blocked senescence via the blocking of several members of the bcl2 family. However, this paper stated: “We found that the MAPT P301S PS19 mouse model of tau-dependent neurodegenerative disease accumulates p16Ink4β-positive senescent astrocytes and microglia”. Thus, their model was that these senescence proteins were in microglia and astrocytes and involved the protein p16. The model presented in this patent application is very different both in the cell type that has the abnormal proteins (neurons) and in the scope of abnormal proteins that have accumulated in these dysfunctional cells. It is the equivalent to a discussion that turning a switch may cause a light to illuminate compared to a discussion of what happens with regards to the circuitry and power sources required for the light bulb to illuminate. In the first case an observation is made. In the second case the basic process and its essential components are discussed that allow one to deduce that altering the power source or the circuitry may affect the light bulbs illumination.

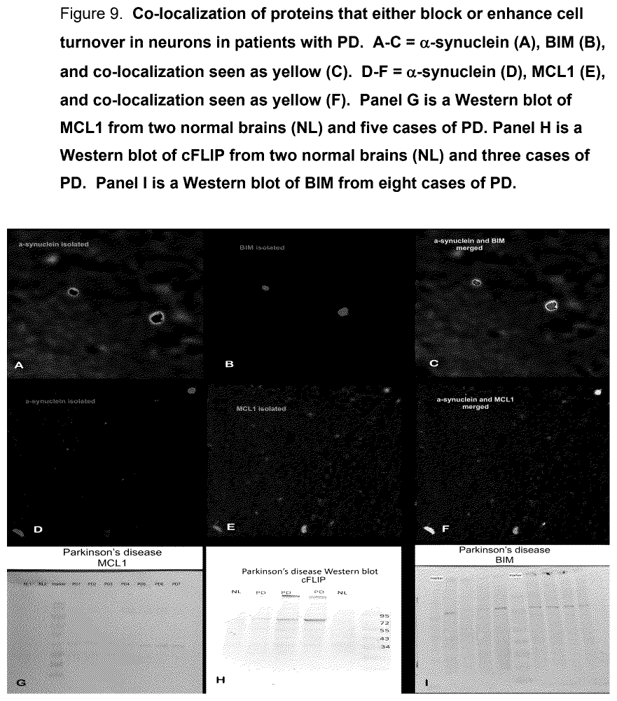

The data from which this patent application is based were derived from different. Inter-locking methodologies. The co-expression data is based on the powerful in situ method which allows one to easily identify not only if a given target RNA or protein is present in the tissue, but in which specific cell type(s). Also used was Western blot hybridization in which the intact proteins are removed from the unfixed tissues and separated via electrophoresis by size on a gel and then detected via hybridization with a target specific antibody (FIG. 9). Also used was qRTPCR which is a highly sensitive PCR based method that not only allows one to determine if a given mRNA target is in a given unfixed tissue, but also, when present, the amount of the mRNA is also obtained. Using frozen tissues from PD we compared the amount of mRNAs from the bcl2 family and cFLIP versus control tissues. Only Parkinson's disease tissues with α-synuclein were included in the study (12/16 samples). For many of the mRNAs there was a highly significant increase in the PD cases as well as the AD cases as seen in the following table:

TABLE 1
Analysis of mRNA levels in PD and AD samples relative
to controls for the bcl2 family and cFLIP.

| mRNA | PD - p value vs controls | AD - p value vs controls |
|---|---|---|
| Bcl2 | <0.00001 | <0.00001 |
| BIM | <0.00001 | <0.00001 |
| MCL1 | 0.002 | <0.00001 |
| BAD | 0.003 | <0.00001 |
| Cflip | 0.001 | <0.00001 |
| bclXL | 0.001 | <0.00001 |
| BAX | 0.002 | <0.00001 |

It should be stressed that these data were derived only from tissues in which either the α-synuclein protein (PD) or hyperphosphorylated tau protein (AD) was detected and that the burden of hyperphosphorylated tau protein was 2× greater per cm² than the amount of α-synuclein which may explain why the p values are lower (ie, greater significance) in the AD versus the PD cases. Interestingly, other mRNAs of the bcl2 family were significantly increased in the AD tissues compared to controls, and when compared to AD tissues that lacked hyperphosphorylated tau protein, that included NOXA, PUMA, BAK, as well as p53. However, neither NOXA (p value 0.048), PUMA (p value 0.08) or BAK (p value 0.42) mRNAs levels in PD were elevated compared to the control tissues which may reflect differences in the pro-apoptotic protein milieu in different neurodegenerative diseases.

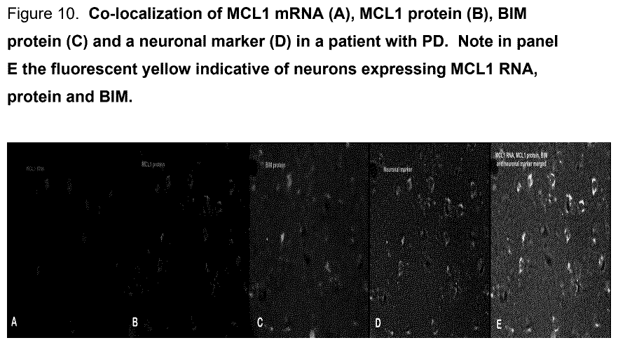

Our group has extensive experience with RNA in situ hybridization. Thus, it was straightforward for us to corroborate the qRTPCR data listed in Table 1 with in situ hybridization on the corresponding formalin fixed, paraffin embedded tissues. This was done for MCL1 and cFLIP mRNA, We indeed noted a marked increase in the number of cells with MCL1 and cFLIP mRNA in the cases of ALS, CBD, Parkinson's disease, and LBD compared to controls. Co-expression analyses provided another layer of documenting the specificity of these data because this documented that the cells with MCL1 mRNA also contained MCL1 protein, BIM protein, and were neurons, as depicted in FIG. 10 in a case of PD.

It is well documented that the bcl2 family of proteins (both inhibitors of cell turnover and activators of cell turnover) are strongly associated with cancers. The reason is that cancer cells define an “immortalized” state in that they have developed mechanisms to avoid cell death. This idea when extrapolated to neurons in neurodegenerative diseases is the foundation of my new, original, and novel theory of what molecular mechanism links the disparate neurodegenerative diseases. Clearly, cancer cells also actively proliferate which is easily demonstrated by markers such as Ki-67 whereas neuronal replacement by stem cells is a much less evident, but still critical process. It is thus not surprising that many anti-cancer drugs have been developed that can block bcl2, bclW, bclXL, cFLIP, and/or MCL1. As one example, the gene MCL1, encoding the antiapoptotic protein MCL1, is frequently duplicated or its expression otherwise amplified in human cancer cells. A number of transcriptional repressors, including anthracyclines, decrease MCL1 expression. As described in Wei, et al., Cancer Cell 21, 547-562 (2012), BCLXL expression confers resistance to transcriptional repressors, such as MCL1 repression. In particular a number of approved anti-cancer drugs are known to have significant dose-related repression of MCL1 expression. These compounds include flavopiridol, triptolide, 5,6-dichlorobenzimidazole riboside (DRB), actinomycin D, the kinase inhibitor 5-iodotubercidin, doxorubicin, daunorubicin, and epirubicin.

A number of compounds are identified as effective at inhibiting cFLIP activity, and thereby releasing cancer cells into a cell turnover pathway, ie apoptotic death. As reviewed by Safa, et al, Cancers, 3, 1639-1671 (2011), such compounds include both transcriptional inhibitors of cFLIP expression and compounds that function at the post-transcriptional level. Compounds identified with inhibition of cFLIP transcription include cisplatin, oxaliplatin, doxorubicin, camptothecin, 9-nitrocamptothecin (9-NC), irinotecan, vorinostat, trichostatin, droxinostat (CMH), valproic acid, celastrol, zerumbone, withaferin A, quinacrine, Lupeol, Chrysin, S-adenosylmethionine, CDDO-imidazolide, and salirasib. Compounds implicated in post-transcriptional effects on c-FLIP activity include Actinomycin D, anisomycin, cyclohexamide, cystamine, eupatolide, 5-fluorouracil (5-FU), genistin, imatinib mesylate, paxilline, rapamycin, rocaglamide, silibinin, sorafenib, Tamoxifen, Taxol (paclitaxel), and Troglitazone. Clearly, the above list of anti-cFLIP and anti-MCL1 molecules that could be effective in the diagnosis and treatment of Alzheimer's disease is not exhaustive. Nonetheless, screening of potential compounds can be greatly enhanced by focus on those compounds that exhibit anti-apoptotic activity in vitro or in a model system such as mice, especially using well defined mouse models of neurodegenerative diseases for AD, PD, and ALS.

Although the blocking of MCL1 and cFLIP are indeed important in cancer therapy and, by extension, in neurodegenerative diseases by allowing the dysfunctional “immortalized” neurons to enter cell turnover and be replaced by healthy neurons, there is a wealth of information with regards to bcl2 inhibition. Bcl2 is an excellent target for cancer therapy because it is not highly expressed in normal tissues and some tumors, such as chronic lymphocytic leukemias, express very high amounts. In comparison, bclW is expressed in high amounts in the normal bone marrow and, thus, blockage of bclW will have many side effects such as anemia, bleeding diathesis, and increased risk of infection. One small molecule that blocks bcl2 very effectively with few side effects is venetoclax and, as expected, is FDA approved as a treatment against several cancers which express high amounts of bcl2. It follows that therapies directed against other bcl2 family members, and/or against the molecules involved in moving such proteins in and out of the nucleus, may be able to play a key role in both diagnosing Alzheimer's disease and other neurodegenerative diseases such as CBD, PD, LDB, ALS, CTE and, critically, serve as the basis for treating these diseases. It should be stressed that blockage of bcl2, bclXL, MCL1 and cFLIP (as well as other proteins that can inhibit the turnover of dysfunctional neurons in neurodegenerative diseases) can be achieved both by the many small molecules already approved for such or by monoclonal antibodies directed against these proteins as well as other strategies.

My group is currently doing experiments with the mouse model of human Alzheimer's disease calledTau4RΔK. These mice express a transgene encoding a mutant four-repeat domain of human microtubule-associated protein tau protein (Q244-E372). The mice express only human hyperphosphorylated tau protein in their brain as well as spinal cord which is evident at about 4 months of age. At about this time they exhibit many different neurological signs and symptoms of dementia as well as motor deficits since the spinal cord is also involved. The mice typically die at around 11-13 months of end-stage dementia with the hyperphosphorylated tau protein at autopsy showing a pattern of expression (cortex, hippocampus, limbic system) equivalent to that seen in end stage human Alzheimer's disease although, as noted, the spinal cord is also involved and, thus, these mice also show motor-related problems which are easy to identify with simple clinical tests.

We obtained eight of these mice in May 2023 at 1 month old and since the end of May five have received venetoclax IP (intraperitoneal) three times a week at the recommended dosage for humans and three received no treatment. The mice are now 4 months old. The three untreated mice are more sluggish/less active than their venetoclax treated litter mates. Further, the untreated mice have a jerky-type gate with wider distances between their hind limbs, indicative of motor problems, when compared to the mice treated with venetoclax that are moving normally The mice were recently subjected to the grip strength test where the variable measured was the length of time before the mouse lost its grip and fell back into the cage. The average time for the three untreated mice was 10 seconds. The average time that the mice treated with venetoclax lost their grip and fell back into the cage was 28 seconds! This is nearly a 3-fold improvement in muscular strength. These mice will be carefully monitored and tested for several more months and then will be sacrificed and extensive molecular testing will be done of the brain for the mRNAs and proteins that form the basis of this grant application.

### Materials and Methodologies Used in this Patent Application

### 1. Patient Samples

The patient samples (formalin fixed paraffin embedded excisional autopsy material or matched frozen tissues) came from stored files obtained with respective IRB approved protocols that de-identified all data except for the diagnosis, age, extent of disease, clinical workup to rule out other possible causes of dementia, and sex of the patient. As of now over 100 tissues of Alzheimer's disease from over 30 patients have been identified in which sections of the cerebral cortex (frontal and temporal lobes) as well as hippocampus plus entorhinal sections were available for review. Negative controls included over 60 tissues from age-matched controls where there was no clinical evidence of Alzheimer's disease. My group also has obtained samples from 20 people with ALS, 16 with PD (which include both formalin fixed, paraffin embedded tissues and frozen tissue), 4 cases of CBD, and 4 cases of LBD. In each case, large (typically 2.0 cm) full thickness sections of the brain tissue were available for analysis. Each case and control was tested by immunohistochemistry testing for hyperphosphorylated tau protein, TFP-43, and α-synuclein to confirm the clinical/pathologic diagnosis.

### 2. Immunohistochemical Analysis

The immunohistochemical protocol has been previously described and uses the Leica BOND-MAX (Leica Biosystems, Buffalo Grove, IL—see reference list above for papers by Nuovo G et al that detail these protocols). As detailed in the Nuovo G J et al peer review publications, the primary antibodies tested were all commercially available and most were obtained from ABCAM, Enzo Life Sciences, and Proteintech Laboratories.

### 3. Co-Expression Analysis

A co-expression analyses protocol was implemented as has been previously described in the applicants' existing publications. The computer-based analysis by the Nuance system (Caliper Life Sciences, Hopkinton, MA) separates each chromogenic spectral signal, converts it to a fluorescent signal, then mixes the two or more signals and indicates if cells contain the two or more targets of interest. Quantification is achieved with the Nuance software.

### 4. In Situ Hybridization Analyses

The in situ hybridization protocol was as previously described in the inventors' existing publications. In brief, in situ hybridization was performed using LNA modified and 5′ digoxigenin tagged probes specific for the MCL1 or cFLIP mRNA. The probe/target complex was visualized after the alkaline phosphatase-linked conjugate reacted with the chromogen, nitroblue tetrazolium and bromochloroindolyl phosphate (NBT/BCIP) with a nuclear fast red counterstain. Negative controls included omission of the probe and the use of a scrambled probe.

5. qRTPCR Analyses

We did qRTPCR for RNA extracted both from frozen tissues and CSF (cerebral spinal fluid). With regards to the tissues, we have thus far analyzed the RNA extracted from over 100 tissues from people with Alzheimer's disease, aged matched controls, and 16 tissues from people with Parkinson's disease. In each case cryostat sections were obtained and fixed in 10% buffered formalin overnight to allow for robust immunohistochemistry. In this matter each tissue could be catalogued as either hyperphosphorylated tau protein positive (AD tissues, neurofibrillary tangles) or phosphorylated a synuclein positive (ie Lewy Body) for PD tissues. All of these qRTPCR analyses for mRNA expression were done at the OSUCCC core lab (by Dr. Paolo Fadda) with GAPDH as the reference gene. They were also done blinded to the clinical/pathologic diagnoses. As seen in Table 1, there was a markedly increased relative expression of cFLIP and many members of the bcl2 family not only between the normal and Alzheimer's disease as well as Parkinson's disease, but importantly in the Alzheimer's disease group when comparing samples+for hyperphosphorylated tau protein versus (the latter data is not shown).

We have also done qRTPCR for a series of CSF samples blinded to the clinical diagnosis. Most of these samples were tested by Dr. Esmerina Tili at OSUCCC. There indeed was a significant increase in the expression of MCL1, cFLIP, and BAX mRNA in the CSF of early and late Alzheimer's disease versus controls. However, I was not satisfied with the amount of RNA obtained from the samples. We thus, in consultation with scientists at Enzo Life Sciences, did a variety of experiments where we spiked normal CSF with varying amounts of MCL1 full length mRNA and tested various kits and extraction protocols. This work clearly showed that the addition of linear acrylamide led to much higher yields of RNA extraction from the CSF and, thus, we have modified our protocol, which will be used to study additional CSF samples.

### 6. Western Blot Analyses

Western blot analyses were done on the AD and PD frozen samples since in our experience these yield much more robust results than proteins obtained from matching formalin fixed paraffin embedded tissues as we have previously published. The same antibodies used for immunohistochemistry were also used for the Western blot analyses which was done using a standard, previously published protocol.

The foregoing descriptions of specific embodiments of the present invention are presented for purposes of illustration and description. The Title, Background, Summary, Detailed Description and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way. Any unintended embracement of such subject matter is hereby disclaimed. They are not intended to be exhaustive nor to limit the invention to precise forms disclosed and, obviously, many modifications and variations are possible in light of the above teaching. The embodiments are chosen and described in order to best explain principles of the invention and its practical application, to thereby enable others skilled in the art to best utilize the invention and its various embodiments with various modifications as are suited to the particular use contemplated. It is intended that a scope of the invention be defined broadly by the Drawings and Specification appended hereto and to their equivalents. Therefore, the scope of the invention is in no way to be limited only by any adverse inference under the rulings of Warner-Jenkinson Company, v. Hilton Davis Chemical, 520 US 17 (1997) or Festo Corp. v. Shoketsu Kinzoku Kogyo Kabushiki Co., 535 U.S. 722 (2002), or other similar caselaw or subsequent precedent should not be made if any future claims are added or amended subsequent to this Patent Application.

We claim:

1. A diagnostic method of assaying any neurodegenerative disease that includes either hyperphosphorylated tau protein (aka neurofibrillary tangles), α-synuclein (aka Lewy bodies), or TDP-43, or a combination thereof in a patient comprising one or more of testing the patient including but not limited to brain tissue, CSF, and blood, for expression of the relevant abnormal protein.

2. The diagnostic method of claim 1, further comprising determining the apoptotic activity in brain tissues or performing a series of assays over time to assess disease progression, wherein the detection of abnormal proteins are associated with the expression of proteins that can block neuronal cell turnover (including but not limited to bcl2, bclW, bclXL, MCL1 and cFLIP) as well as proteins that can promote neuronal cell turnover (including but not limited to, BAD, BIM, PUMA, NOXA (and their regulator p53), BAK, BAX, p16, plus the molecules involved in the nuclear import/export of these target proteins, determining the apoptotic activity in brain tissue or performing a series of such assays over time to determine disease progression.

3. A method for treating neurodegenerative disease in a patient diagnosed with AD, CBD, CTE, PD, LBD, FTD, ALS and related diseases comprising administering a neuronal turnover-inducing drug/compound/biologic in a pharmaceutically effective amount.

4. The method of claim 4, wherein the drug/compound/biologic include small molecules already approved for other diseases as well as monoclonal antibodies capable of either blocking bcl2, bclW, bclXL, cFLIP, and/or MCL1 or equivalent proteins/mRNAs or enhancing the action of proteins/mRNAs that can induce the dysfunctional neurons to enter the neuronal turnover pathway.

5. The treatment of claim 2 wherein the patient is a human patient.

6. The treatment of claim 2 wherein the drug is one or more of an MCL1 inhibitor, a cFLIP inhibitor, a bcl2 inhibitor, or a modulator of other members of the bcl2 family, or a modulator of importin or exportin family members, or a modulator of other drugs/biologics that can effect neuronal turnover.

7. The treatment of claim 4 wherein the patient is a human patient.

8. The method of claim 1 further comprising a system for in vivo testing of drugs for the neurodegenerative diseases including but not limited to AD, CBD, CTE, PD, LBD, FTD, ALS.

9. A method for surveying patients comprising assays for identifying patients that exhibit inhibition of neuronal cell turnover relating to increased expression by proteins/mRNAs including but not limited to bcl2, bclW, bclXL, cFLIP and MCL-1 plus other members of the bcl2 family as well as proteins/mRNAs that often accompany and block these anti-neuronal turnover molecules including but not limited to BAD, BIM, NOXA, PUMA, BAK, BAX, p53, and p16.

10. The method of claim 7 further comprising patients that are one or more of mammals, rodents, mice, rats, primates, human, tissues, and cultured cells.

11. A method of identifying drugs useful in the therapy of neurodegenerative disease comprising selecting drugs with one or more of the activities comprising neuronal cell turnover induction, increase miRNA 512 activity, inhibit bcl2, bclW, bclXL, cFLIP, and/or MCL-1 activity, modulating bcl2 family member activity, modulating importin/exportin family member activity.

12. The diagnostic method of claim 1 applied to other neurodegenerative diseases that include dysfunctional neurons that have accumulated an abnormal protein(s) including but not limited to hyperphosphorylated tau protein, TDP-43, and/or α-synuclein.

13. The diagnostic method of claim 1, further comprising any microRNA or non-coding RNA that has the ability to modulate (either increased or decreased) the various mRNAs and proteins so described in this patent application, including but not limited to MCL1, cFLIP, bcl2 family members, p53, importins, exportins, and modulators of tau protein.

14. The diagnostic method of claim 1, further comprising any mRNA and/or protein that has the ability to modulate (either increased or decreased) the various mRNAs and proteins described in this patent application, including but not limited to MCL1, cFLIP, bcl2 family members, p53, importins, exportins, and modulators of tau protein.
